(19) United States
(12) Patent Application Publication
(10) Pub. No.: US 2008/0159958 A1
(43) Pub. Date: Jul. 3, 2008

(54) DETERMINATION OF HISTAMINE-3 BIOACTIVITY

(75) Inventors: Richard J. Radek, Green Oaks, IL (US); R. Scott Bitner, Pleasant Prairie, WI (US); Marlon D. Cowart, Round Lake Beach, IL (US); Jorge D. Brioni, Vernon Hills, IL (US); Timothy A. Esbenshade, Schaumburg, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 11/950,560

(22) Filed: Dec. 5, 2007

Related U.S. Application Data
(60) Provisional application No. 60/877,275, filed on Dec. 27, 2006.

(51) Int. Cl. A61K 49/00

(52) U.S. Cl. 424/9.2

(57) ABSTRACT

The invention relates to an in vivo method for determining the bioactivity of chemical compounds as histamine-3 receptor (H₃R) ligands, and provides animal models to determine such bioactivity. The invention further relates to methods for screening therapeutic compounds demonstrating a desired property, using such methods and models described.

### CROSS-REFERENCE SECTION TO RELATED APPLICATIONS

This application claims the benefit of U.S. Provisional Patent Application No. 60/877,275, filed Dec. 27, 2006, which is hereby incorporated by reference in its entirety.

### TECHNICAL FIELD OF THE INVENTION

The invention relates to an in vivo method for determining the bioactivity of chemical compounds as histamine-3 receptor (H₃R) ligands, and provides animal models to determine such bioactivity. The invention further relates to methods for screening therapeutic compounds demonstrating a desired property, using such methods and models described.

### DESCRIPTION OF RELATED TECHNOLOGY

Attention deficit hyperactivity disorder (ADHD) is one of the most common familial neurological disorders in children, see for example, Timothy E. Wilens, et al., “Attention deficit/hyperactivity disorder across the lifespan”, Annu. Rev. Med. (2002) 53:113-31. Stimulants such as methylphenidate, amphetamine, and dextroamphetamine have been the principal pharmacological treatment for ADHD for the past 25 years (Spencer T. J., et al., “Novel treatments for attention-deficit/hyperactivity disorder in children”, J. Clin. Psychiatry, (2002) 63 Suppl. 12:16-22). Stimulants increase frontal cortex dopamine by inhibiting catecholamine reuptake, an effect that may underlie the efficacy of the stimulant class of compounds. Although considered as a first line treatment for ADHD, stimulants are ineffective for some patients, and for others, adverse side-effects, such as tics, loss of appetite, and insomnia limit their use (Wilens et al., 2002) Additionally, evidence of abuse liability has led the U.S. FDA to schedule such stimulant compounds, adding further concern over the use of stimulants in children. Atomoxetine (commercially available as STRATTERA®) is the first new drug approved for the treatment of ADHD in over twenty years. Atomoxetine blocks the reuptake of norepinephrine and dopamine in the pre-frontal cortex of rats (Bymaster F. P., et al., “Atomoxetine increases extracellular levels of norepinephrine and dopamine in prefrontal cortex of rat: a potential mechanism for efficacy in attention deficit/hyperactivity disorder”, Neuropsychopharmacology (2002) November 27(5):699-711). While atomoxetine may have fewer side effects than traditional stimulant ADHD medications, it is not as efficacious as methylphenidate and, if effective at all, it sometimes requires titration over several weeks to obtain a desired therapeutic effect (Joseph Biederman, et al., “A Post Hoc Subgroup Analysis of an 18-Day Randomized Controlled Trial Comparing the Tolerability and Efficacy of Mixed Amphetamine Salts Extended Release and Atomoxetine in School-Age Girls with Attention-Deficit/Hyperactivity Disorder”, Clinical Therapeutics (2006) 28(2):280-293; Christopher J. Kratochvil, et al., “An Open-Label Trial of Tomoxetine in Pediatric Attention Deficit Hyperactivity Disorder. Journal of Child and Adolescent”, Psychopharmacology (June 2001) 11:2, 167-170). Certain antidepressants and antipyschotics have been tried, but have only shown limited utility in treating ADHD because of unacceptable side effects or poor efficacy (Joshua Caballero, et al., “Atomoxetine Hydrochloride for the Treatment of Attention-Deficit/Hyperactivity Disorder”, Clinical Therapeutics (2003) 25(12):3065-3083). Thus, efforts continue toward the development of more efficacious, safer, and non-scheduled compounds to treat ADHD (Spencer et al, 2002). An example of compounds thought to be safer and more efficacious are those that target regulation of the central nervous system neurotransmitter histamine, especially through the histamine-3 receptor subtype (H₃R). In particular, H₃R antagonists have been reported as candidates for treatment of neuro-cognitive disorders.

In addition to being beneficial in patients with ADHD, histamine H₃R antagonists are candidates to be effective in treating other central nervous system (CNS) diseases with clinical signs of inattention, memory loss, learning deficits, and cognitive deficits. Some of these include the dementias (e.g. Ahlzheimer's Disease), mild cognitive impairment; and cognitive deficits and dysfunction associated with psychiatric disorders such as schizophrenia, bipolar disorder, depression, drug abuse, mood alteration, obsessive-compulsive disorder, Tourette's syndrome, and Parkinson's disease. Other potential therapeutic uses for H₃R antagonists in nervous system-related diseases and disorders include epilepsy, seizures, pain, neuropathic pain, neuropathy, sleep disorders, narcolepsy, pathological sleepiness, jet lag, motion sickness, dizziness, Meniere's disease, vestibular disorders, vertigo, and obesity. H₃R antagonists are also thought to be useful in several immune system, metabolic, and oncologic conditions, including diabetes, type II diabetes, Syndrome X, insulin resistance syndrome, metabolic syndrome, medullary thyroid carcinoma, melanoma, and polycystic ovary syndrome, allergic rhinitis, and asthma.

Examples of reviews of the benefits of H₃R antagonists in ADHD models or other disease models can be found in Esbenshade, Fox, and Cowart “Histamine H₃R antagonists: Preclinical promise for treating obesity and cognitive disorders” Molecular Interventions (2006) vol 6, pp. 77-88 and Celanire S, Wijtmans M, Talaga P, Leurs R. de Esch J. P. Histamine H₃R antagonists reach for the clinic. Drug Disc Today (2005), vol.10, pp. 1613-1627.

Examples of reports of benefits of H₃R antagonists in ADHD models or other CNS disease models can be found in the following references: Cowart, et al. J. Med. Chem. (2005), vol. 48, pp. 38-55; Fox, G. B., et al. “Pharmacological Properties of ABT-239: II. Neurophysiological Characterization and Broad Preclinical Efficacy in Cognition and Schizophrenia of a Potent and Selective Histamine H₃ Receptor Antagonist”, Journal of Pharmacology and Experimental Therapeutics (2005) 313, 176-190; “Effects of histamine H₃ receptor ligands GT-2331 and ciproxifan in a repeated acquisition avoidance response in the spontaneously hypertensive rat pup.” Fox, G. B., et al. Behavioural Brain Research (2002), 131(1,2), 151-161; Yates, et al. JPET (1999) 289, 1151-1159 “Identification and Pharmacological Characterization of a Series of New 1H-4-Substituted-Imidazoyl Histamine H₃ Receptor Ligands”; Ligneau, et al. Journal of Pharmacology and Experimental Therapeutics (1998), 287, 658-666; Tozer, M. Expert Opinion Therapeutic Patents (2000) 10, p. 1045; M. T. Halpern, “GT-2331” Current Opinion in Central and Peripheral Nervous System Investigational Drugs (1999) 1, pages 524-527; Shaywitz et al., Psychopharmacology, 82:73-77 (1984); Dumery and Blozovski, Exp. Brain Res., 67:61-69 (1987); Tedford et al., J. Pharmacol. Exp. Ther., 275:598-604 (1995); Tedford et al., Soc. Neurosci. Abstr., 22:22 (1996); and Fox, et al., Behav. Brain Res., 131:151-161 (2002); Glase, S. A., et al. “Attention deficit hyperactivity disorder: pathophysiology and design of new treatments.” Annual Reports in Medicinal Chemistry (2002), 37 11-20; Schweitzer, J. B., and Holcomb, H. H. “Drugs under investigation for attention-deficit hyperactivity disorder” Current Opinion in Investigative Drugs (2002) 3, p. 1207.

The neurotransmitter histamine plays a very important role in the regulation of the arousal-sleep continuum. Histaminergic projections from the hypothalamic tuberomammillary nucleus (TBN) to the brainstem locus coeruleus can lead to the activation of the noradrenergic-driven reticular activating system (Barbara E. Jones, “From waking to sleeping: neuronal and chemical substrates” Trends in Pharmacological Sciences (2005) 26(11):578-86). The ascending reticular activating system not only plays an important role in sleep-wake homeostasis, but neuronal projections of this system to the pre-frontal cortex are thought to be equally important for processes of attention and vigilance (Paus T., “Functional anatomy of arousal and attention systems in the human brain”, Prog. Brain Res. (2000) 126:65-77).

The histamine H₃R is located at multiple sites in the CNS. H₃ receptors on histaminergic nerve terminals function as autoreceptors, and regulate the release of histamine. Histamine H₃R antagonists induce the release of the histamine, which can bind to and stimulate histamine H₁ and H₂ receptors. In this way, histamine H₃R antagonists can stimulate increased histaminergic synaptic activity that promotes attention (Cowart, et al. J. Med. Chem. (2005), vol. 48, pp. 38-55; Fox, G. B., et al. J. Pharmacol. Exp. Therapeutics (2005) 313, 176-190).

In contrast to the attention and cognition promoting properties of histamine H₃R antagonists, antagonism of the postsynaptic H₁R is known to produce CNS sedation, as well as impairment of cognitive performance. The H₁R antagonist diphenhydramine has been widely studied in both animals (doses ranging 10-100 mg/kg) and humans (doses ranging 25-100 mg). At doses used for over-the-counter formulations (50 mg), diphenhydramine has been reported to produce cognitive impairment in humans, as well as electroencephalographic (EEG) signs of sedation and drowsiness (Alan Gevins, Michael E. Smith, and Linda K. McEvoy, “Tracking the Cognitive Pharmacodynamics of Psychoactive Substances with Combinations of Behavioral and Neurophysiological Measures”, Neuropsychopharmacology (2002) 26(1):29-39). Electroencephalograms are clinically measured brain wave potentials that accurately assess states of low arousal, drowsiness and sleep, as well as wakefulness and arousal. EEG signs of low arousal, drowsiness and sleep generally correspond to states of inattention, low vigilance, and poor cognitive function, while EEG signs of wakefulness and arousal are associated with attention and vigilance. Low frequency slow waves are one of the EEG potentials associated with sedation, sleep, and drowsiness, and are augmented by H₁ antagonists such as diphenhydramine. The EEG and cognitive effects of H₁ receptor antagonists have been well characterized in humans, and also in animal species such as the rat (Y. Kaneko, et al., “The Mechanism Responsible for the Drowsiness Caused by First Generation H1 Antagonists on the EEG Pattern”, Methods Find Exp. Clin. Pharmacol. (2000) 22(3): 163-168; Kamei C., et al., “Influence of certain H1-blockers on the step-through active avoidance response in rats”, Psychopharmacology. (1990) 102(3):312-8; Saitou K., et al., “Slow wave sleep-inducing effects of first generation H1-antagonists”, Biol. Pharm. Bull. (1999) 22(10):1079-82).

Animal models that reliably predict H₃R antagonist activity and efficacy in humans would greatly benefit the process of developing H₃R antagonists as therapeutic agents to treat CNS diseases such as ADHD. Of particular use would be an animal model that measures an H₃R antagonist effect that is very similar to effects that would be predicted to occur in humans.

Accordingly, it would be beneficial to provide methods for determining the bioactivity of histamine H₃R ligands, particularly H₃R antagonists, in a cost-effective and efficient manner in animal models and in humans, such that the research and development of more promising therapeutic compounds of this mechanism would be greatly enhanced. Such methods would improve the process of evaluation of histamine H₃R antagonist clinical candidates, and thereby enhance the development of such compounds as more efficacious, safer, and non-scheduled pharmaceutical agents.

### SUMMARY OF THE INVENTION

In one aspect, the invention relates to a method for detecting H₃R antagonist activity, efficacy, or both, in an animal model. The method relates to the ability of histamine H₃R antagonists to reduce, block, attenuate, reverse, or partially reverse EEG activity produced by an H₁ antagonist in a test animal.

In particular, the method relates to administering a histamine-1 receptor (H₁R) antagonist of sufficient dosage to an animal to produce a change in the recorded EEG, for example, a dose that increases low frequency slow wave EEG amplitude; and administering a H₃R antagonist in the same animal to determine, or identify, a dose or doses that reduces or decreases the effects of the H₁R antagonist on EEG. More particularly, the H₃R antagonist can attenuate, block, reverse, or partially reverse the effects of the H₁R antagonist on EEG.

The method is particularly useful in assessing whether the compound is an H₃R agent that is effective, particularly an H₃R antagonist that is effective in vivo. The data obtained from the method can be interpreted and accordingly can be correlated to effects that would likely be seen in human clinical trials. The data obtained would be particularly beneficial in the design and conduct of clinical trials in humans.

In another aspect, the invention provides an in vivo means for assessing H₃R antagonist activity, H₃R antagonist efficacy, or both H₃R antagonist activity and efficacy, or lack thereof, comprised of administering an H₁R antagonist of sufficient dosage to an animal to produce a change in the recorded EEG, for example, a dose that increases low frequency slow wave EEG amplitude; and administering a H₃R antagonist in the same animal to identify a H₃R antagonist dose or doses that reduce, attenuate, block, reverse, or partially reverse the effects of the H₁R antagonist on EEG.

Accordingly, the invention provides an animal model for assessing histamine-3 activity, efficacy, or both, of a test compound, in a preclinical setting. The data obtained would be particularly beneficial in determining whether the test compound demonstrates desired properties of H₃R antagonist, efficacy, or both, to further provide a suitable pharmaceutical agent.

Another aspect of the invention relates to an assay, or means, for identifying an H₃R antagonist that is that demonstrates H₃R antagonist activity, H₃R antagonist efficacy, or both H₃R antagonist activity and efficacy, or lack thereof, particularly in vivo, comprising administering a desired test compound to an animal and demonstrating that the desired test compound can decrease the effects of H₁R antagonists on brain wave potentials. The means or assay is particularly useful when the brain potential activity of the animal is recorded by electroencephalography and the animal demonstrates a change in EEG activity induced by an H₁R antagonist when recorded via electroencephalography.

In particular, such assay or means can be accomplished by administering a histamine H₁R antagonist of sufficient dosage to an animal to produce a change in the recorded EEG, for example, a dose that increases low frequency slow wave EEG amplitude; and administering a desired test compound, which can include an H₃R antagonist, in the same animal to identify a dose or doses that reduces or decreases the effects of the H₁R antagonist on brain wave potentials, for example, such that the test compound attenuates, blocks, reverses, or partially reverses the effects of the H₁R antagonist on EEG. Such identified compounds can be provided for further testing, as well as pre-clinical development, or further clinical development, as necessary and desired, to provide pharmaceutical compounds for treating H₃ receptor related disorders or conditions.

As such, the invention relates to a method for identifying a H₃R agent, comprising the steps of: a) measuring the EEG in an animal and establishing a dose of an H₁R antagonist that changes brain wave potentials; b) measuring the EEG in an animal and establishing a dose of an H₃R antagonist that does not change relevant brain wave activity; c) co-administering an H₁R antagonist and H₃R antagonist to an animal at doses established in a) and b) above; and d) measuring and analyzing the EEG to determine whether the effects of the H₁R antagonist on brain wave potentials have been blocked, attenuated, partially reversed, or reversed. The method is particularly when the animal is compared to brain wave potentials in the same animal when treated with vehicle only, i.e., under conditions of placebo treatment. Such method is useful for identifying a H₃R antagonist.

The method is particularly useful in a clinical setting wherein the brain wave potentials are compared in a human subject. For example, the brain wave potentials in a human subject administered H₁R antagonist treatment, H₃R antagonist treatment, or both, are compared with the brain wave potentials of a human subject when treated with vehicle only. In this aspect, the invention relates to a method for assessing activity of an H₃R agent in a human subject, comprising the steps of: a) measuring the EEG in the subject and establishing a dose of an H₁R antagonist that changes brain wave potentials; b) measuring the EEG in a subject and establishing a dose of a H₃R antagonist that does not change such brain wave activity; c) co-administering a H₁R antagonist and H₃R antagonist to a subject at doses established in a) and b) above; and d) measuring and analyzing the EEG to determine whether the effects of the H₁R antagonist on brain wave potentials have been reduced or decreased, such that it is determined that the brain wave potentials have been blocked, attenuated, partially reversed, or reversed.

Such means and methods and further means and methods contemplated as part of the invention are further described herein.

### BRIEF DESCRIPTION OF THE FIGURES

FIG. 1. Compound 1, (3aR, 6aR)-2-[4′-(5-Methyl-hexahydro-pyrrolo[3,4-b]pyrrol-1-yl)-biphenyl-4-yl]-2H-pyridazin-3-one (1.0 mg/kg i.p.) and thioperamide (30.0 mg/kg i.p.) lower 1-4 Hz amplitude for the first hour after injection. ABT-239 did not produce significant effects to lower 1-4 Hz amplitude at the doses tested. The data is expressed as a percent change from vehicle control (placebo) treatment. *p<0.05 vs. vehicle control. One way repeated measures ANOVA, Newman-Keuls post-tests.

FIG. 2. The histamine H₁R antagonist diphenhydramine (10.0 mg/kg i.p.) significantly increased 1-4 Hz amplitude for the first hour after injection. The data is expressed as a percent change from vehicle control (placebo) treatment. *p<0.05 vs. vehicle control. One way repeated measures ANOVA, Newman-Keuls post-tests.

FIG. 3. Compound 1, (3aR, 6aR)-2-[4′-(5-Methyl-hexahydro-pyrrolo[3,4-b]pyrrol-1-yl)-biphenyl-4-yl]-2H-pyridazin-3-one (0.03-0.1 mg/kg i.p.) significantly reduced the effect of diphenhydramine to increase of 1-4 Hz amplitude. The data is expressed as a percent change from vehicle control (placebo) treatment. *p<0.05 vs. diphenhydramine. One way repeated measures ANOVA, Newman-Keuls post-tests.

FIG. 4. ABT-239 (0.3 mg/kg i.p.) significantly reduced the effect of diphenhydramine to increase of 1-4 Hz amplitude. The data is expressed as a percent change from vehicle control (placebo) treatment. *p<0.05 vs. diphenhydramine. One way repeated measures ANOVA, Newman-Keuls post-tests.

FIG. 5. Thioperamide (3.0 mg/kg i.p.) significantly reduced the effect of diphenhydramine to increase of 1-4 Hz amplitude. The data is expressed as a percent change from vehicle control (placebo) treatment. *p<0.05 vs. diphenhydramine. One way repeated measures ANOVA, Newman-Keuls post-tests.

### DETAILED DESCRIPTION OF THE INVENTION

### Abbreviations and Terms

As used herein, the term “electroencephalography” refers to a technique of measuring electrical potentials (activity) of the brain, also referred to as brain waves or brain wave potentials.

As used herein, the term “electroencephalograp” refers to equipment used for measuring brain wave potentials.

As used herein, the term “electroencephalogram” refers to brain waves or brain wave potentials. This term can also refer to the data generated by the electroencephalograph.

As used herein, the term “record, recorded, or recording” refers to the use of laboratory instruments and techniques to measure biological activity, in this case, the electroencephalogram.

As used herein, “EEG” denotes an abbreviation of electroencephalography, electroencephalograph, or electroencephalogram.

As used herein, the term “co-administering” refers to the process of injecting two substances into an animal or human, with no inference as to the order, dosage, route of administration, or timing of the injections.

### Animals

The animal can be any suitable mammal for assessing brain wave potentials, and in particular, can be humans, primates, or rodents. Examples of suitable rodents are rats, mice, hamsters, guinea pigs, and the like. Suitable primates are suitable, including humans, monkeys, baboons, and the like. Non-rodent animals also are suitable, and can include, for example, cattle, horses, pigs, sheep, goats, cats, dogs, and the like.

### Compounds and Methods

A suitable H₁R antagonist is one that can augment EEG activity associated with sedation, sleep, or drowsiness in animals and humans. Particularly preferred are those H₁R antagonists considered pharmaceutically effective and safe for human use. Examples of such H₃R antagonists include, but are not limited to, chlorpheniramine, brompheniramine, diphenhydramine, pyrilamine, and tripelennamine. A particularly suitable H₁R antagonist is diphenhydramine.

Suitable test compounds can be any chemical compound suitably administered to an animal or human. In one embodiment, the method provides H₃R antagonists or candidates. Confirmatory analysis can be carried out using a recognized H₃R agent, particularly H₃R antagonist, and more particularly those H₃R antagonists considered pharmaceutically efficacious and safe for human use. Accordingly, as used herein, the term “histamine-3 receptor agent” is a compound demonstrating, or having been identified as a compound having, H₃R related activity, for example, a H₃R ligand, particularly H₃R antagonists. As used herein, the terms ‘histamine H₃R antagonist’, ‘histamine-3 receptor antagonist’, and ‘H₃R antagonist’ encompass and describe compounds that prevent receptor activation by an H₃R agonist alone, such as histamine; it also encompasses compounds known as ‘inverse agonists’. H₃R inverse agonists are compounds that not only prevent receptor activation by an H₃R agonist, such as histamine, but also inhibit intrinsic H₃R activity.

Examples of such H₃R antagonistinclude, but are not limited to the following: thioperamide; ABT-239 (4-{2-[2-((R)-2-Methyl-pyrrolidin-1-yl)-ethyl]-benzofuran-5-yl}-benzonitrile); (3aR, 6aR)-2-[4′-(5-Methyl-hexahydro-pyrrolo[3,4-b]pyrrol-1-yl)-biphenyl-4-yl]-2H-pyridazin-3-one; A-349821; ABT-834; A-688057 (4-{2-[2-((R)-2-Methyl-pyrrolidin-1-yl)-ethyl]-benzofuran-5-yl}-1H-pyrazole); ciproxifan; BF-2649 (Ciproxidine, 1-(3-(3-(4-chlorophenyl)propoxy)propyl)piperidine, Schwartz, et al. European Patent application EB 0982300(A2); JNJ-17216498; JNJ-10181457; JNJ-5207852; JNJ-6379490; GSK-189254A (6-(3-Cyclobutyl-2,3,4,5-tetrahydro-1H-benzo[d]azepin-7-yloxy)-N-methyl-nicotinamide, Wilson, D. The discovery of a novel series of potent, orally active histamine H₃R antagonists. 13th Royal Society of Chemistry Medicinal Chemistry Symposium. Cambridge, UK, Sept. 4-7 2005).

More particularly, examples of suitable H₃R antagonists include, but are not limited to: thioperamide; ABT-239 (4-(2-[2-((R)-2-Methyl-pyrrolidin-1-yl)-ethyl]-benzofuran-5-yl}-benzonitrile); (3aR, 6aR)-2-[4′-(5-Methyl-hexahydro-pyrrolo[3,4-blpyrrol-1-yl)-biphenyl-4-yl]-2H-pyridazin-3-one; ABT-834; A-688057 (4-{2-[2-((R)-2-Methyl-pyrrolidin-1-yl)-ethyl]-benzofuran-5-yl}-1H-pyrazole); ciproxifan; BF-2649 (Ciproxidine, 1-(3-(3-(4-chlorophenyl)propoxy)propyl)piperidine;JNJ- 17216498; JNJ-10181457; JNJ-5207852; JNJ-6379490; GSK-189254A (6-(3-Cyclobutyl-2,3,4,5-tetrahydro-1H-benzo[d]azepin-7-yloxy)-N-methyl-nicotinamide.

More particularly still, suitable histamine H₃R antagonists include thioperamide, 4-{2-[2-((R)-2-Methyl-pyrrolidin-1-yl)-ethyl]-benzofuran-5-yl}-benzonitrile (ABT-239), and 3aR, 6aR)-2-[4′-(5-methyl-hexahydro-pyrrolo[3,4-b]pyrrol-1-yl)-biphenyl-4-yl]-2H-pyridazin-3-one (Compound 1).

Assessment and identification of the data can be based on any standardized measurement of EEG brain wave potential. The EEG represents the measurement of electrical potentials produced by the brain. The EEG can be used for classifying pharmacological agents and evaluating their pharmacodynamics. Quantitative EEG analysis reveals distinct wave profiles across pharmacological classes that include neuroleptics, antidepressants, hypnotics, tranquilizers, nootropic/cognition-enhancing drugs, and psychostimulants (Saletu B., et al., “Classification and evaluation of the pharmacodynamics of psychotropic drugs by single-lead pharmaco-EEG, EEG mapping and tomography (LORETA)” Methods Find. Exp. Clin. Pharmacol. (2002) 24(Suppl C):97-120). Similar pharmacological EEG profiles have been demonstrated between species, in particular rat and human. Specifically, drug-induced changes in low frequency EEG amplitude, which also can be referred to as delta and slow wave activity, can be used to distinguish between drugs that either depress or stimulate CNS activity in both rat and human (Porsolt RD, et al., “New perspectives in CNS safety pharmacology” Fundam. Clin. Pharmacol. (2002) 16(3):197-207; Sannita W. G., “Quantitative EEG in human neuropharmacology Rationale, history, and recent developments” Acta Neurol. (Napoli) (1990) 12(5):389-409. Increased amplitude of low frequency EEG is associated with drowsiness, sleep, inattention, and low vigilance. Low frequency EEG amplitude can be detected, identified, and analyzed by several objective and subjective methods that are widely accepted in the field. Among quantitative analyses, the fast fourier transform (FFT) method is often used to determine the predominant amplitude and frequency of the EEG signal. The frequency band of slow wave activity determined by FFT analysis is sometimes reported, but not limited to, the range of about 1 Hertz (Hz) to about 4 Hz. Any subjective or objective method regarded in the field as being accurate for identifying low frequency EEG (e.g., slow waves, delta activity), or any other EEG pattern associated with drowsiness, sleep, inattention, or low vigilance, could be used to detect the ability of H₃R antagonists to counteract the effects of H₁R antagonists.

One with skill in the art, who is knowledgeable in the methods of evaluating EEG data, would be able to assess and identify the EEG profiles to determine whether the patterns are sufficiently similar or different to provide guidance on the H₃R activity of a desired compound. For example, one with skill might assess a change in recorded brain potential in an animal treated with a H₁R antagonist and determine that a particular dose of H₃R antagonist decreases low frequency EEG amplitude in such a manner as to attenuate, block, reverse, or partially reverse the effects of the H₁R antagonist on EEG. However, further guidance is provided in the illustrations and examples that follow.

### EXAMPLES

The invention is further described and illustrated by way of the following examples and experimental details provided therein. The examples are intended to aid the understanding of the invention are not to be construed as a limitation of the invention in any way.

### Example Compounds

Compound 1 is (3aR, 6aR)-2-[4′-(5-methyl-hexahydro-pyrrolo[3,4-b]pyrrol-1-yl)-biphenyl-4-yl]-2H-pyridazin-3-one, which is further described in Reference Example A below.

Compound 2 is ABT-239, also known as 4-(2-[2-((R)-2-methyl-pyrrolidin-1-yl)-ethyl]-benzofuran-5-yl}-benzonitrile, Chemical Abstracts registry number 460746-46-7, reported in Cowart, et al. Journal of Medicinal Chemistry (2005), vol. 48, pp. 38-55.

Compound 3 is thioperamide, N-cyclohexyl-4-(1H-imidazol-4-yl)piperidine-1-carbothioamide, Chemical Abstracts registry number 106243-16-7.

### Reference Example A

### (3aR, 6aR)-2-[4′-(5-Methyl-hexahydro-pyrrolo[3,4-b]pyrrol-1-yl)-biphenyl-4-yl]-2H-pyridazin-3-one

### Step 1: (3aR, 6aR)-5-Methyl-hexahydro-pyrrolo[3,4-b]pyrrole-1-carboxylic acid tert-butyl ester

(3aR, 6aR)-Hexahydro-pyrrolo[3,4-b]pyrrole-1-carboxylic acid tert-butyl ester (CAS # 370880-09-4) may be prepared as described in Schenke, T., et al, “Preparation of 2,7-Diazabicyclo[3.3.0]octanes”, U.S. Pat. No. 5,071,999, published Dec. 10, 1991, which provides a racemate which may be resolved by chromatography on a chiral column or by fractional crystallization of diasteromeric salts, or as described in Basha, et al. “Substituted Diazabicycloalkane Derivatives”, U.S. Patent Publication No. 2005/101602, published May 12, 2005.

To a solution of (3aR, 6aR)-hexahydro-pyrrolo[3,4-b]pyrrole-1-carboxylic acid tert-butyl ester (18.31 g, 0.86 mol) in methanol (450 ml) was added paraformaldehyde (52 g, 1.72 mole) and the mixture was stirred at room temperature for 1 hour. Sodium cyanoborohydride was then added and the mixture was stirred at room temperature for 10 hours, diluted with 1N NaOH (450 ml), extracted with dichloromethane (5×200 ml). The combined organic layers were dried (Na₂SO₄), filtered and concentrated to provide the title compound. ¹H NMR (300 MHz, DMSO-d₆) δ ppm 4.18 (m, 1 H) 3.47-3.59 (m, 1 H) 3.34-3.46 (m, 2 H) 2.75-2.90 (m, 1 H) 2.71 (m, 1 H) 2.44-2.60 (m, 2 H) 2.29 (s, 3 H) 1.89-2.06 (m, 1 H) 1.65-1.81 (m, 1 H) 1.42-1.49 (m, 9 H). MS: (M+H)¹⁹ =226.

### Step 2: (3aR, 6aR)-5-Methyl-hexahydro-pyrrolo[3,4-b]pyrrole

To a solution of (3aR, 6aR)-5-Methyl-hexahydro-pyrrolo[3,4-b]pyrrole-1-carboxylic acid tert-butyl ester (20.8 g, 0.86 mole) in methanol (450 ml) was added aqueous 3N HCl (300 ml). The mixture was stirred at room temperature overnight, then concentrated to dryness at 30° C. under vacuum. The residue was treated with aqueous 1N NaOH to obtain a pH of 9-10. The mixture was concentrated to dryness. The crude material was purified by chromatography (eluting with a mixture of 10% methanol and 1% ammonium hydroxide in dichloromethane) to provide the title compound. ¹H NMR (300 MHz, CDCl₃) δ ppm 4.12-4.17 (m, 1 H) 3.31-3.43 (m, 1 H) 3.19-3.30 (m, 1 H) 3.12 (d, J=11.53 Hz, 1 H) 2.88-3.01 (m, 1 H) 2.69 (dd, J=9.49, 2.37 Hz, 1 H) 2.40-2.52 (m, 2 H) 2.33 (s, 3 H) 2.12-2.28 (m, 1 H) 1.82-1.95 (m, 1 H). MS: (M+H)⁺=127.

### Step 3: (3aR, 6aR)-1-(4-Bromo-phenyl)-5-methyl-octahydro-pyrrolo[3,4-b]pyrrole

A mixture of (3aR, 6aR)-5-Methyl-hexahydro-pyrrolo[3,4-b]pyrrole, 4,4′-dibromobiphenyl (1.15 eq), tris(dibenzylideneacetone)dipalladium (0.2 equivalents), racemic-2,2′-bis(diphenylphosphino)-1,1′-binaphthyl (0.4 equivalents) and sodium tert-butoxide (1.5 equivalents) were dissolved in 1 ml/equivalent of toluene and heated to 70° C. under N₂ for overnight. The mixture was cooled to room temperature, diluted with water and extracted with dichloromethane (5×). The combined organics were dried over sodium sulfate, filtered and concentrated and purified by chromatography (eluting with a mixture of 5% methanol in dichloromethane) to provide the title compound. ¹H NMR (300 MHz, CDCl₃) δ ppm 7.39-7.53 (m, 6 H) 6.60-6.66 (m, 2 H) 4.17-4.23 (m, 1 H) 3.52-3.61 (m, 1 H) 3.26-3.35 (m, 1 H) 2.98-3.05 (m, 1 H) 2.70-2.80 (m, 2 H) 2.58-2.64 (m, 2 H) 2.38 (s, 3 H) 2.15-2.26 (m, 1 H) 1.97 (m, 1 H). MS: (M+H)⁺=357/359.

### Step 4: (3aR, 6aR)-2-[4′-(5-Methyl-hexahydro-pyrrolo[3,4-b]pyrrol-1-yl)-biphenyl-4-yl]-2H-pyridazin-3-one

A mixture of (3aR, 6aR)-1-(4-Bromo-phenyl)-5-methyl-octahydro-pyrrolo[3,4-b]pyrrole (4.54 g, 12.6 mmole), 3(2H)-pyridazinone (2.41 g, 25.2 mmole), copper powder (1.60 g, 25.2 mmole) and potassium carbonate (5.21 g, 37.7 mmole) were dissolved in 63 ml of quinoline and heated at 150° C. under N₂ for 48 hours. The mixture was cooled to room temperature, diluted with hexane (15 ml) and filtered through diatomaceous earth. The filtrate was concentrated under reduced pressure and the residue was purified by chromatography (eluting first with diethyl ether, followed by dichloromethane, then elution with a mixture of 5% methanol in dichloromethane) to provide the title compound. ¹H NMR (300 MHz, CDCl₃) δ ppm 7.91 (dd, J=3.73, 1.70 Hz, 1 H) 7.61-7.65 (m, 4 H) 7.51 (d, J=8.48 Hz, 2 H) 7.25 (dd, dd, J=9.40, 4.07 Hz, 1 H) 7.07 (dd, J=9.49, 1.70 Hz, 1 H) 6.64 (d, J=8.81 Hz, 2 H) 4.19-4.27 (m, 1 H) 3.54-3.64 (m, 1 H) 3.28-3.38 (m, 1 H) 3.00-3.11 (m, 1 H) 2.56-2.85 (m, 4 H) 2.40 (s, 3 H) 2.10-2.29 (m, 1 H) 1.89-2.05 (m, J=6.78 Hz, 1 H); MS (M+H)⁺=373. The solid (3aR, 6aR)-2-[4′-(5-methyl-hexahydro-pyrrolo[3,4-b]pyrrol-1-yl)-biphenyl-4-yl]-2H-pyridazin-3-one obtained showed a melting range of 204-207 ° C. (dec.).

### Determination of in Vitro Potency at Histamine H₃ Receptors

To determine the effectiveness of representative compounds of this invention as H₃ receptor ligands, the following tests were conducted according to previously described methods (see European Journal of Pharmacology, 188:219-227 (1990); Journal of Pharmacology and Experimental Therapeutics, 275:598-604 (1995); Journal of Pharmacology and Experimental Therapeutics, 276:1009-1015 (1996); and Biochemical Pharmacology, 22:3099-3108 (1973)).

The rat and human H₃ receptor was cloned and expressed in cells, and competition binding assays carried out, according to methods previously described (see Esbenshade, et al. Journal of Pharmacology and Experimental Therapeutics, vol. 313:165-175, 2005; Esbenshade et al., Biochemical Pharmacology 68 (2004) 933-945; Krueger, et al. Journal of Pharmacology and Experimental Therapeutics, vol. 314:271-281, 2005. Membranes were prepared from C6 or HEK293 cells, expressing the rat histamine H₃ receptor, by homogenization on ice in TE buffer (50 mM Tris-HCl buffer, pH 7.4, containing 5 mM EDTA), 1 mM benzamidine, 2 μg/ml aprotinin, 1 μg/ml leupeptin, and 1 μg/ml pepstatin. The homogenate was centrifuged at 40,000 g for 20 minutes at 4° C. This step was repeated, and the resulting pellet was resuspended in TE buffer. Aliquots were frozen at −70° C. until needed. On the day of assay, membranes were thawed and diluted with TE buffer.

Membrane preparations were incubated with [³H]-N-α-methylhistamine (0.5-1.0 nM) in the presence or absence of increasing concentrations of ligands for H₃ receptor competition binding. The binding incubations were conducted in a final volume of 0.5 ml TE buffer at 25° C. and were terminated after 30 minutes. Thhoperamide (30 μM) was used to define non-specific binding. All binding reactions were terminated by filtration under vacuum onto polyethylenimine (0.3%) presoaked Unifilters (Perkin Elmer Life Sciences) or Whatman GF/B filters followed by three brief washes with 2 ml of ice-cold TE buffer. Bound radiolabel was determined by liquid scintillation counting. For all of the radioligand competition binding assays, IC₅₀ values and Hill slopes were determined by Hill transformation of the data and pKᵢ values were determined by the Cheng-Prusoff equation. Kᵢ values are converted from the pKᵢ values according to Kᵢ=10^{(-pKi)}. Compounds 1, 2, and 3 are histamine H₃R antagonists, with high potency at H₃ receptors. The table below shows the potencies in competition binding assays as Ki values.

| | rat | human |
|---|---|---|
| | H₃ | H₃ |
| | Ki | Ki |
| | (nM) | (nM) |
| Compound 1, ((3aR,6aR)-2-[4′-(5-Methyl- | 8.1 | 1.9 |
| hexahydro-pyrrolo[3,4-b]pyrrol-1-yl)-biphenyl- | | |
| 4-yl]-2H-pyridazin-3-one) | | |
| Compound 2, ABT-239, (4-{2-[2-((R)-2-Methyl- | 0.45 | 1.35 |
| pyrrolidin-1-yl)-ethyl]-benzofuran-5-yl}- | | |
| benzonitrile) | | |
| Compound 3, thioperamide, (N-cyclohexyl-4-(1H- | 3.6 | 72 |
| imidazol-4-yl)piperidine-1-carbothioamide) | | |

### General Methods

I. Subjects

All experiments have been approved by the Institutional Animal Care and Use Committee (IACUC) at Abbott Laboratories and are in strict accordance with the ethical guidelines for use of laboratory animals. All experiments were conducted with male adult CD-1 rats of the Sprague-Dawley strain (Charles River Laboratories, Portage, Mich.) with body weights in the range of 400-600 g. When the rats were not in the laboratory being tested, they were housed 1 per cage in a climate controlled room with 12 hour lights on, 12 hour lights off cycle and food provided ad-lib.

II. Surgery

For anesthesia during surgical implantation of EEG recording electrodes, rats are administered Nembutal (Abbott Laboratories) 50 mg/ml ip. After achieving a deep, stable plane of anesthesia, scalp hair is removed using electric clippers and the rat is placed into the ear and incisor bars of a stereotaxic instrument to immobilize the head. The scalp is disinfected with povidone iodine, and an incision is placed longitudinally along the midline of the scalp and the tissue retracted from the skull with a blunt probe. EEG recording electrodes are bilaterally implanted over the parietal (−2.0 mm anterior-posterior, 4.0 mm lateral from bregma) and frontal (+2.0 mm anterior-posterior, 3.0 mm lateral from bregma) cortices. A reference electrode was placed 11.0 mm posterior to bregma along the centerline (0.0 mm lateral). Cortical surface electrodes consist of stainless steel screws (size #90-00) soldered to a fine wire and a miniature electrical socket. To implant the electrodes, small holes are drilled (#60 bit) into the skull, taking care not to damage the dura with the drill bit. The surface electrodes are screwed into the holes to a depth that comes in contact with, but does not penetrate the dura covering the brain. Once in place, the electrodes along with the miniature connector are permanently affixed to the skull with acrylic dental cement. The rats are given a 10-14 day recovery period from the surgery before experiments are conducted.

III. EEG Recordings

The EEG was recorded from rats inside sound-attenuating chambers (Med Associates Inc, St. Albans, Vt.). Before any pharmacological experiments began, implanted rats were habituated to the EEG recording chambers for 2-5 hours on 5 consecutive days. When placed into the recording chambers, a flexible cable is attached to the miniature connector implanted on the rats. This cable allows the rat unrestricted movement within the chambers during the recording session. EEG amplifiers (AM Systems, Inc., Carlsborg, Wash.) and a computer-based data acquisition system (Datawave Inc., Berthoud, Colo.) were used to acquire (256 Hz sampling rate) and analyze data. All experiments and habituation sessions were conducted during the light phase of the circadian cycle.

IV. Drug Studies

A. Effects of H₁ and H₃ Receptor Antagonists

Dose response effects on EEG were determined for the H₃R antagonist Compound 1 (0.01-1.0 mg/kg), ABT-239 (0.1-3.0 mg/kg), and thioperamide (3.0-30.0 mg/kg). The selected doses for these compounds are in the range that enhance cognition, but do not disrupt exploratory motor activity or motor coordination. Dose response effects on EEG were also determined for the H₁ antagonist diphenhydramine (1.0-10.0 mg/kg). The doses selected for diphenhydramine are within the range that disrupts cognition, but do not disrupt exploratory motor activity or motor coordination. Each rat received a vehicle control treatment (placebo), and all doses of the test compounds. All treatments were administered by the intraperitoneal (i.p.) route of administration. The treatments were administered in a random order on different days with one treatment per day, and at least 2 days between treatments. This within subjects design allowed each rat to serve as its own control. EEG recordings were begun within 10 minutes after injection and recording sessions lasted for 120 minutes. The time of day for injections and subsequent recordings were between 10:00 AM and 2:00 PM.

IV. Drug Studies

B. Effects of Co-Administering H₁ and H₃ Receptor Antagonists

Each rat received 4 different treatments on separate days, each treatment being a combination of two injections. The treatment groups are listed in Table 1. The first injection was administered 15 minutes before the second injection. The EEG recordings were begun within 10 minutes after this second injection. All treatments were administered by the intraperitoneal (i.p.) route of administration. The treatments were administered in random order across days with at least two days between treatments. Again, each rat served as its own control. The EEG recording sessions lasted for 120 minutes. The time of day for injections and subsequent recordings were between 10:00 AM and 2:00 PM.

TABLE 1

| | Injection 1 | Injection 2 |
|---|---|---|
| Treatment 1 | Vehicle (placebo) | Vehicle (placebo) |
| Treatment 2 | H₃R Antagonists | Vehicle (placebo) |
| | 1. Compound 1 (0.01-0.1 mg/kg) | |
| | or, | |
| | 2. ABT-239 (0.3 mg/kg) | |
| | or, | |
| | 3. Thioperamide (3.0 mg/kg) | |
| Treatment 3 | Vehicle (placebo) | Diphenhydramine |
| | | (10.0 mg/kg) |
| Treatment 4 | H₃R Antagonists | Diphenhydramine |
| | 1. Compound 1 (0.01-0.1 mg/kg) | (10.0 mg/kg) |
| | or, | |
| | 2. ABT-239 (0.3 mg/kg) | |
| | or, | |
| | 3. Thioperamide (3.0 mg/kg) | |

V. Analysis of EEG

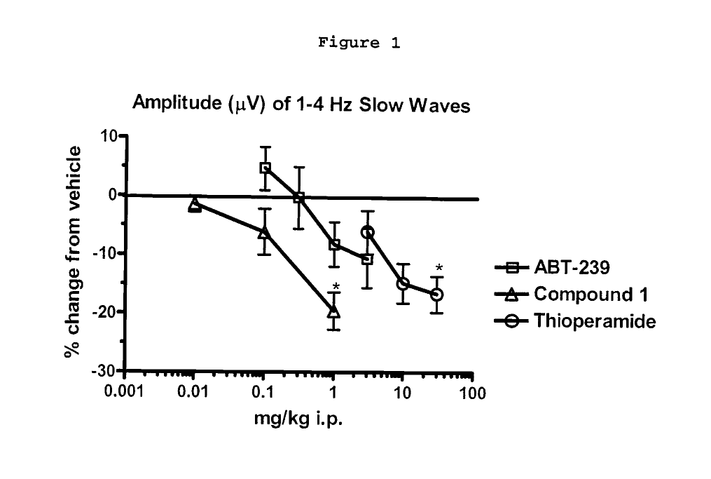

Assessment of cortical low frequency EEG amplitude in the 1-4 Hz band (delta) was used as an electrophysiological measure of H₁R and H₃R antagonist activity in rats. The average 1-4 Hz EEG amplitude in microvolts (μV) was determined for 10 second epochs using Fast Fourier Transform (FFT) analysis. To determine the average 1-4 Hz EEG amplitude for the first 60 minutes of the recording, 360-10 sec FFT analyzed epochs were averaged together. Epochs that contained movement artifact in the EEG were excluded from this averaging (<5% of all epochs). A repeated measure, one-way ANOVA was utilized for statistical evaluation of average FFT data with treatment as the repeated measure. A Newman-Keuls hoc test was used for comparisons between treatments. The average 1-4 Hz amplitude data for the first hour of EEG recording is graphically expressed (FIGS. 1-5) as a percent change from vehicle control values.

VI. Drug Preparation

All doses are expressed in mg/kg of free base of the compounds. Diphenhydramine and thioperamide were purchased from Sigma Chemical Company (St. Louis, Mo.). Compound 1 and ABT-239 were synthesized at Abbott Laboratories. For use, Compound 1, ABT-239, and thioperamide were dissolved in sterile water-1% citric acid solution (pH ˜5.3). The sterile water-1% citric acid solution served as the vehicle control (placebo) treatment for the H₃Rantagonists (injection 1). Diphenhydramine was dissolved in a sterile 0.9% NaCl solution (pH ˜5.5). The sterile 0.9% NaCl solution served as the vehicle control (placebo) treatment the H₁ antagonist diphenhydramire.

### Evaluation of Data

FIG. 1 shows that the non-imidazole H₃R antagonist Compound 1 (1.0 mg/kg) and imidazole H₃R antagonist thioperamide (30.0 mg/kg) lower the average amplitude of 1-4 Hz EEG in rats for a period of 1 hour after injection. This effect, also termed EEG activation, is consistent with the promotion of wakefulness and has been previously reported in the literature for the H₃R antagonists thioperamide and ciproxifan (Ligneau et al 1998, Lin et al, 1990). The lower doses of thioperamide (3.0-10. mg/kg) and Compound 1 (0.01-0.1 mg/kg) did not produce significant lowering of 1-4 Hz EEG amplitude. Another non-imidazole H₃R antagonist compound, ABT-239 (0.1-3.0 mg/kg), did not produce statistically significant lowering of 1-4 Hz EEG slow waves. However, a trend toward a decrease was observed at the 3.0 mg/kg dose, consistent with the wake promoting effects observed with other H₃R antagonists.

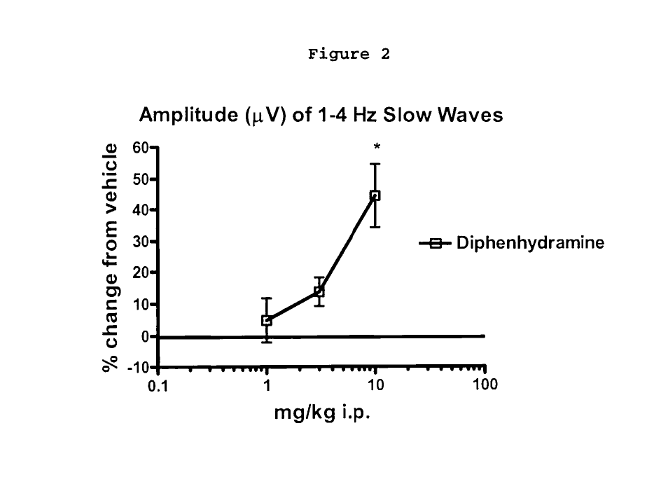

FIG. 2 shows the effects of the H₁R antagonist diphenhydramine on rat 1-4 Hz EEG amplitude. In contrast to H₃R antagonists, diphenhydramine (10.0 mg/kg) significantly increased average amplitude of 1-4 Hz EEG. This is consistent with the well-known sedative or drowsiness producing effects of widely used over-the-counter anti-histamine drugs for allergies (Turner C., et al., “Sedation and memory: studies with a histamine H-1 receptor antagonist”, J. Psychopharmacol. (2006) 20(4):506-17). The two lower doses of diphenhydramine (1.0-3.0 mg/kg) were not significantly different from vehicle control.

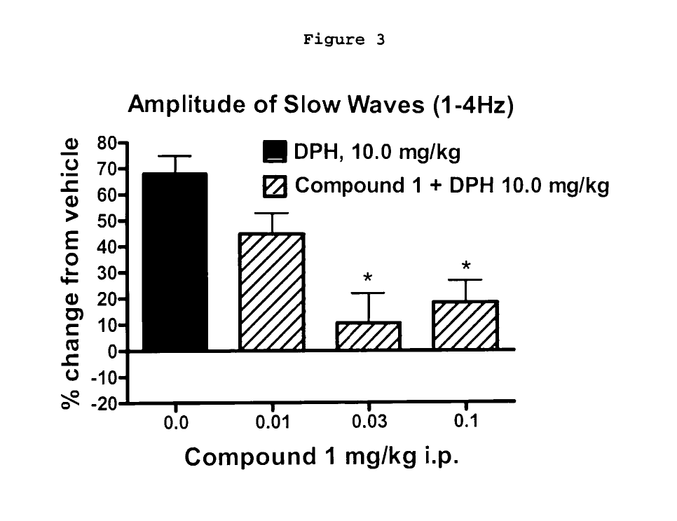

FIG. 3 shows the effects of the H₃R antagonist Compound 1 on increased 1-4 Hz amplitude produced by the H₁R antagonist diphenhydramine. Pre-treatment of rats with Compound 1 (0.03 mg/kg and 0.1 mg/kg) significantly reduces diphenhydramine (10.0 mg/kg) induced increases of average 1-4 Hz EEG amplitude. The low dose of Compound 1 (0.01 mg/kg) produced a trend toward reducing the effects of diphenhydramine, however, this did not achieve statistical significance.

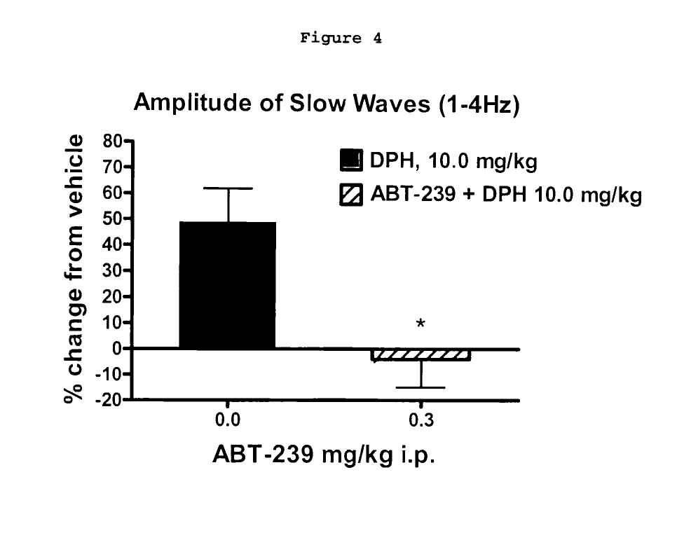

FIG. 4 shows the effects of another non-imidazole H₃R antagonist ABT-239 on diphenhydramine EEG. Like Compound 1, ABT-239 (0.3 mg/kg) significantly reduces the effects of diphenhydramine (10.0 mg/kg) on 1-4 Hz EEG amplitude. At the doses that reduced the effect of diphenhydramine on EEG, neither Compound 1 nor ABT-239 had effects on the EEG when administered alone (see FIG. 1).

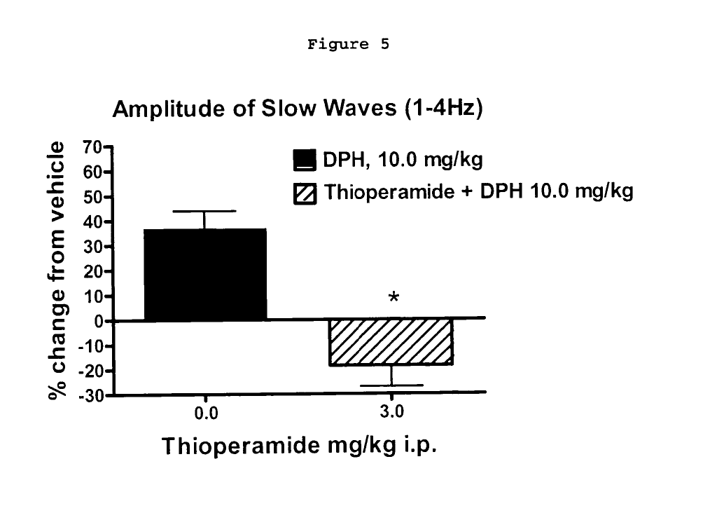

FIG. 5 shows the effects of the imidazole H₃R antagonist thioperamide on diphenhydramine-induced increases of slow wave amplitude. Like the non-imidazoles, thioperamide (3.0 mg/kg) significantly reduces the effects of diphenhydramine (10.0 mg/kg). Furthermore, the dose of thioperamide that reduced diphenhydramine effects did not have significant effects on the EEG when administered alone (see FIG. 1).

As demonstrated by the Examples above, the H₃R antagonists Compound 1, ABT-239, and thioperamide indeed attenuate or reduce the increase in 1-4 Hz EEG amplitude produced by the H₁R antagonism of diphenhydramine. The ability to demonstrate H₃R antagonist activity was dependent on selecting a dose of the H₁R antagonist diphenhydramine (10.0 mg/kg) that had an effect on the EEG by itself, namely, in this case, increasing the average amplitude of 1-4 Hz low frequency EEG. The magnitude of diphenhydramine effects at the 10 mg/kg dose used to demonstrate an H₃R antagonist effect in these examples ranged from about 38% to about 68%. The reduction of diphenhydramine-induced effects on EEG by H₃R antagonists was seen with two major chemotypes, both imidazole and non-imidazole. The doses of Compound 1 (0.03-0.1 mg/kg), ABT-239 (0.3 mg/kg), and thioperamide (3.0 mg/kg) that attenuated the effects of diphenhydramine did not have significant effects on the EEG when administered alone, suggesting a pharmacological interaction rather than a summation of opposing physiological effects of the H₃R antagonists combined with the H₁R antagonists. Moreover, in addition to blocking the effects of diphenhydramine on EEG, 0.3 mg/kg of ABT-239 is within the range of doses that improves learning and memory performance in rodents (Fox G.B., et al., “Pharmacological properties of ABT-239 (4-(2-{2-[(2R)-2-Methylpyrrolidinyl]ethyl)-benzofuran-5-yl)benzonitrile]: II. Neurophysiological characterization and broad preclinical efficacy in cognition and schizophrenia of a potent and selective histamine H3 receptor antagonist”, J. Pharmacol. Exp. Ther. (2005) 313(1):176-90. Thus, blocking the effects of diphenhydramine on the rodent EEG by H₃R antagonists is predictive of the doses that improve cognitive function in rodents.

Diphenhydramine has well known effects to produce learning and memory deficits in rodents, and clinically relevant cognitive impairment in humans (Mansfield L., et al., “Effects of fexofenadine, diphenhydramine, and placebo on performance of the test of variables of attention (TOVA)”, Ann Allergy Asthma Immunol. 90(5):554-9; Taga C., et al., “Effects of vasopressin on histamine H(1) receptor antagonist-induced spatial memory deficits in rats”, Eur. J Pharmacol. (2001) 6;423(2-3):167-70). It is widely accepted that EEG neurophysiology, as well as drug effects on the EEG, are highly conserved across mammalian species, including between rodent and human. Diphenhydramine, for example, produces increases in human low frequency EEG similar to those reported in our studies with rats (Givens et al 2002). Since cortical EEG can readily be measured in humans, and diphenhydramine has well established human EEG effects, the ability of H₃R antagonists to counteract the effects diphenhydramine could be tested clinically (Oken B. S., “Pharmacologically induced changes in arousal: effects on behavioral and electrophysiologic measures of alertness and attention”, Electroencephalogr. Clin. Neurophysiol. (1995) 95(5):359-71). In such case, the animal model provides a highly useful pre-clinical biomarker to 1) predict human plasma levels needed to produce H₃R antagonist activity, and 2) predict doses needed to achieve improvement of cognitive function in humans. Compounds that do not block diphenhydramine in rodents, or another suitable animal, pre-clinically, would not advance to be tested in expensive clinical efficacy trials.

Histamine is an endogenous excitatory neurotransmitter in the mammalian central nervous system. H₃ receptors are thought to act as autoreceptors, thus, H₃R activation is thought to reduce presynaptic release of histamine (Arrang J. M., et al., “Autoregulation of histamine release in brain by presynaptic H3-receptors”, Neuroscience (1985) 15(2):553-62). Conversely, blocking the H₃ receptor with an H₃R antagonist increases histamine release (Tedford C. E., et al., “Pharmacological characterization of GT-2016, a non-thiourea-containing histamine H3 receptor antagonist: in vitro and in vivo studies”, J. Pharmacol. Exp. Ther., (1995) 275(2):598-604). H₃R antagonists, by blocking feedback inhibition, would increase histamine availability to the post-synaptic membrane. The net effect would be to produce increased activation of the central nervous system, an effect seen with high doses of H₃R antagonists on the rat EEG. At non-activating, low doses of the H₃R antagonists, histamine release may still result in occupancy of significant numbers of post-synaptic histamine receptors. This occupancy may be sufficient enough to compete with diphenhydramine mediated histamine receptor blockade and prevent diphenhydramine drowsiness. Therefore, besides being a potentially useful clinical biomarker, H₃R antagonist reversal of diphenhydramine, or another suitable H₁R antagonist, in animals, could be a useful as bioassay that reliably identifies compounds with H₃R antagonist pharmacology in vivo.

In summary, we describe a potentially useful pharmacological rodent model to test H₃R antagonists by reversing H₁R antagonist-induced changes in rat EEG. This model takes advantage of the high correspondence between rodent and human EEG to predict clinical efficacy and H₃R activity of H₃R antagonists.

Those skilled in the art will recognize, or be able to ascertain using no more than routine experimentation, numerous equivalents to the specific method and reagents described herein, including alternatives, variants, additions, deletions, modifications, and substitutions. Such equivalents are considered to be within the scope of this invention and defined by the following applications.

What is claimed is:

1. A method for evaluating a test compound comprised of administering a histamine-1 receptor (H₁R) antagonist of sufficient dosage to an animal to produce a change in the recorded brain wave potentials associated with an increased low frequency electroencephalography (EEG) amplitude; and administering a histamine-3 receptor (H₃R) antagonist in the same animal to determine a dose that decreases the effects of the H₁R antagonist on EEG.

2. The method of claim 1, wherein the H₁R antagonist is cholorpheniramine, brompheniramine, pyrilamine, or tripelennamine.

3. The method of claim 1, wherein the H₁R antagonist is diphenhydramine.

4. The method of claim 1, wherein the brain wave potential of the animal is measured by electroencephalography that demonstrates the H₃R antagonist attenuates, blocks, reverses, or partially reverses the effects of the H₁R antagonist.

5. The method of claim 4, wherein the electroencephalograph assesses low frequency slow wave patterns at about 1 Hz to about 4 Hz.

6. The method of claim 1, wherein the animal is a human, primate, or rodent.

7. A means of assessing H₃R antagonist activity, H₃R antagonist efficacy, or both H₃R antagonist activity and efficacy, or lack thereof, of a test compound, by administering a desired test compound to an animal and demonstrating that the desired test compound can decrease the effects of H₁R antagonists on brain wave potentials.

8. The means of claim 7, wherein the animal is a human, primate, or rodent.

9. The means of claim 8, wherein the animal is a human.

10. The means of claim 7, wherein the brain potential activity of the animal is recorded by electroencephalography and the animal demonstrates a change in EEG activity induced by an H₁R antagonist when recorded via electroencephalography.

11. The means of claim 10, wherein a desired test compound is administered to the animal and the EEG activity is assessed for whether the test compound attenuates, blocks, reverses, or partially reverses the effects of the H₁R antagonist in low frequency slow wave brain potential activity.

12. The means of claim 11, wherein the low frequency slow wave brain potential activity is determined to be a frequency of from about 1 Mz to about 4 Hz.

13. A method for identifying a H₃R agent, comprising the steps of: a) measuring the EEG in an animal and establishing a dose of an H₁R antagonist that changes brain wave potentials; b) measuring the EEG in an animal and establishing a dose of an H₃R antagonist that does not change relevant brain wave activity; c) co-administering an H₁R antagonist and H₃R antagonist to an animal at doses established in a) and b) above; and d) measuring and analyzing the EEG to determine whether the effects of the H₁R antagonist on brain wave potentials have been blocked, attenuated, partially reversed, or reversed.

14. The method of claim 13, wherein the change in brain wave potential of the animal is compared to brain wave potentials in the same animal under condition of placebo treatment.

15. The method of claim 14, wherein the H₃R agent is a H₃R antagonist.

16. The method of claim 15, wherein the H₃R antagonist is thioperamide; ABT-239 (4-{2-[2-((R)-2-methyl-pyrrolidin-1-yl)-ethyl]-benzofuran-5-yl}-benzonitrile); (3aR, 6aR)-2-[4′-(5-methyl-hexahydro-pyrrolo[3,4-b]pyrrol-1-yl)-biphenyl-4-yl]-2H-pyridazin-3-one; ABT-834; A-688057 (4-(2-[2-((R)-2-methyl-pyrrolidin-1-yl)-ethyl]-benzofuran-5-yl}-1H-pyrazole); ciproxifan; BF-2649 (Ciproxidine, 1-(3-(3-(4-chlorophenyl)propoxy)propyl)piperidine; JNJ-17216498; JNJ-10181457; JNJ-5207852; JNJ-6379490; or GSK-189254A (6-(3-Cyclobutyl-2,3,4,5-tetrahydro-1H-benzo[d]azepin-7-yloxy)-N-methyl-nicotinamide.

17. The method of claim 15, wherein the H₃R antagonist is thioperamide, ABT-239, or Compound 1 (3aR, 6aR)-2-[4′-(5-methyl-hexahydro-pyrrolo[3,4-b]pyrrol-1-yl)-biphenyl-4-yl]-2H-pyridazin-3-one.

18. A method for assessing activity of an H₃R agent in a human subject, comprising the steps of: a) measuring the EEG in a human subject and establishing a dose of an H₁R antagonist that changes brain wave potentials; b) measuring the EEG in a human subject and establishing a dose of an H₃R antagonist that does not change relevant brain wave activity; c) co-administering an H₁R antagonist and H₃R antagonist to a human subject at doses established in a) and b) above; and d) measuring and analyzing the EEG to determine whether the effects of the H₁R antagonist on brain wave potentials have been reduced.
